(19) United States
(12) Patent Application Publication
(10) Pub. No.: US 2023/0329979 A1
(43) Pub. Date: Oct. 19, 2023

(54) COMPOSITIONS COMPRISING RADIOPAQUE SUBSTANCES WITH IMPROVED BIOABSORBABILITY

(71) Applicant: Maruchi Co., Ltd., Wonju-si Gangwon-do (KR)

(72) Inventors: Sung Wook JANG, Seoul (KR); Yun Sung Jang, Seoul (KR)

(73) Assignee: Maruchi Co., Ltd., Wonju-si Gangwon-do (KR)

(21) Appl. No.: 18/212,784

(22) Filed: Jun. 22, 2023

Related U.S. Application Data
(63) Continuation of application No. PCT/KR23/03053, filed on Mar. 6, 2023.

(30) Foreign Application Priority Data
Mar. 4, 2022 (KR) 10-2022-0028333

(51) Int. Cl. A61K 6/54; A61K 6/853; A61K 6/69; A61K 6/78

(52) U.S. Cl. CPC A61K 6/54; A61K 6/853; A61K 6/69; A61K 6/78

(57) ABSTRACT

Provided is a biostable radiopaque substance that is absorbed within living tissue or inflammatory lesion in living tissue and uses thereof. The radiopaque substance does not cause an inflammatory reaction in surrounding tissue and is rapidly absorbed before healing occurs, thereby not interfering with healing and regeneration of damaged tissue. Therefore, the radiopaque substance of the present disclosure can be included in a biotransplantation material, for example, a root canal filling material for endodontic treatment, and be effectively used.

### CROSS-REFERENCE TO RELATED APPLICATION

This application is a continuation of International Application No. PCT/KR2023/003053 filed on Mar. 6, 2023, which claims priority to Korean Patent Application No. 10-2022-0028333 filed on Mar. 4, 2022, the disclosure of which is incorporated herein by reference in its entirety.

### TECHNICAL FIELD

The present disclosure relates to a radiopaque substance with improved bioabsorbability and uses thereof. Specifically, the present disclosure relates to a radiopaque substance with improved bioabsorbability and dental uses thereof. The radiopaque substance of the present disclosure does not cause an inflammatory reaction in surrounding tissue and is rapidly absorbed before healing occurs, thereby not interfering with healing and regeneration of damaged tissue.

### BACKGROUND ART

Various techniques are combined and used in medicine for the treatment of diseases. One of such techniques is X-ray imaging which enables accurate observation of an area where a medical technique has been applied. In order to accurately read radiographic results for a location of a procedure material or an area where it has been applied, a sufficient amount of a radiopaque substance needs to be included in a composition intended for in vivo application.

In addition, since such a radiopaque substance helps to observe a healing process after procedure and a degree of disappearance of an absorbable composition, most countries require that at least a certain amount of a radiopaque substance be included in a composition entering the body.

Recently, substances with nano-sized particles, which are bioinert or biocompatible and radiopaque, have been developed. Research is being conducted on the purpose of increasing radiopacity by adding, to a polymer, a substance composed mainly of BaSO₄, gold, iodixanol, Fe₃O₄, or the like. However, using a radiopaque substance of nanoparticles in a way in which it is added to a polymer or attached to an implantation material may cause a problem in that the radiopaque substance of nanoparticles leaks out when the implantation material is absorbed or leaks as the surface of the implantation material is peeled off. In particular, in vivo safety of the radiopaque substance of nanoparticles has not yet been established when it leaks due to problems such as galvanic corrosion.

Specific examples of a medical field, in which such a radiopaque substance is used, include endodontic treatment that is a branch of dentistry. Endodontic treatment, also known as root canal treatment, is performed in a case where pulp tissue is infected due to tooth fracture or decay, and refers to a process of removing pulp tissue, disinfecting root canal, and then filling and sealing it with a filling material.

Temporary root canal filling materials (which are also referred to as “temporary intracanal medicaments” and used for chemical therapy to assist in removal and disinfection of pulp tissue in such a manner that the material is usually placed in a tooth for about a week and then removed) or permanent root canal filling materials (which are permanently present in root canal and used for the purpose of sealing an empty space inside the root canal so that there is no space for bacteria to grow), which are used in the course of such endodontic treatment, contain a radiopaque substance for a surgeon to ensure that the material has been applied accurately to a desired area.

The temporary or permanent root canal filling material needs to be placed as deeply as possible into every nook and cranny of root canal. To achieve this, excessive pressure is applied, which often causes a case where the root canal filling material is pushed out of accessory canal or apical foramen. In addition, such a case may occur in the course of attempting to disinfect even major foramen during procedure (this is called apical foramen enlargement), and may also occur when there is external apical root resorption or when apical foramen is too large. In addition, the root canal filling material may go outside root canal in a case where a surgeon intentionally applies excessive pressure to make a puff or to fill accessory canal. When the root canal filling material is pushed out of root canal, it is quite difficult to physically remove it again.

Temporary root canal filling materials using calcium hydroxide mainly contain barium sulfate as a radiopaque substance. In general, barium sulfate is evaluated as safe. However, many studies have shown that barium sulfate has relatively high cytotoxicity as compared with other radiopaque substances. In particular, it is also pointed out that an overfilled temporary root canal filling material is a cause of adverse effects. Moreover, even barium sulfate, which is known to have relatively high bioabsorbability, is problematic in that it remains on an applied area for a long time and acts as a defect in normal healing or regeneration of tissue.

Zirconium oxide, which is recently used as a radiopaque substance for temporary root canal filling materials using calcium hydroxide due to these problems, is evaluated as safe because it is inert in vivo and non-cytotoxic. However, zirconium oxide is hardly absorbed by tissue around root apex and remains for a long time, thereby acting as a defect in normal healing or regeneration of tissue.

In addition, there are cases where a root canal filling material is exposed outside root canal over time due to benign tumor, supernumerary tooth, external resorption caused by impacted wisdom tooth, external resorption occurring during orthodontic treatment, or the like. The root canal filling material exposed outside root canal of a tooth remains for a long time and interferes with formation of normal hard tissue such as alveolar bone.

In addition, in a case where a root canal filling material is injected into an area where a dental root needs to mature in the course of treating an immature permanent tooth, the root canal filling material causes a problem of interfering with a normal dental root formation process. This problem occurs not only in an immature permanent tooth but also in a case where a dental root is fractured due to trauma. Healing occurs as hard tissue such as dental root and alveolar bone regenerates in the fractured area; however, a radiopaque substance contained in the root canal filling material, which is injected for the purpose of healing, is not absorbed and remains for a long time, thereby interfering with normal healing and regeneration of hard tissue.

In addition, for a calcium silicate sealer, which has recently been attracting attention as a root canal filling material, there is a problem in that in the course of injecting the sealer or pushing in a GP cone after injecting the sealer, a lot of the sealer goes beyond apical foramen and is injected into apical lesions. Even if the calcium silicate sealer goes beyond apical foramen, it is rare to have an adverse effect on healing of apical lesions. However, in a case where an excessive amount of the calcium silicate sealer is injected into maxillary sinus, inferior alveolar canal, mental foramen, or the like, a radiopaque substance contained therein may rarely cause an adverse effect such as maxillary sinusitis and paresthesia, or in a case where the injected calcium silicate sealer remains therein for a long time, the radiopaque substance may also be misleading in medical judgment when a problem arises later due to another cause.

In many literatures, it has been reported that metal oxides, such as radiopaque substances, are easily removed by macrophages in most of the human body even if their particle size exceeds 100 microns. However, in the area of root apex or alveolar bone around a dental root, metal oxides are not absorbed for a long time and remain therein even in a case where there is a lesion. In particular, it is believed that metal oxides remain for a longer period of time in a case where normal healing occurs so that inflammation is eliminated and macrophages disappear.

Therefore, in order to provide a root canal filling material that does not interfere with normal healing and regeneration after treatment, there is a need for a radiopaque substance with both bioabsorbability and biocompatibility.

### SUMMARY

### Technical Problem

An object of the present disclosure is to solve all of the above-described problems.

An object of the present disclosure is to provide a radiopaque substance with improved bioabsorbability.

Another object of the present disclosure is to provide a composition comprising a radiopaque substance that is rapidly absorbed in living tissue such as living soft tissue or lesion and does not interfere with normal healing and regeneration of tissue.

Yet another object of the present disclosure is to provide a temporary or permanent root canal filling material composition comprising a radiopaque substance that is rapidly absorbed before healing occurs and does not interfere with healing and regeneration of damaged tissue.

Still yet another object of the present disclosure is to provide a method for applying, to a subject, a composition comprising a radiopaque substance with improved bioabsorbability.

Still yet another object of the present disclosure is to provide a medical or dental use of a radiopaque substance with improved bioabsorbability.

The objects of the present disclosure are not limited to those mentioned above. The objects of the present disclosure will become more apparent from the following description, and will be realized by means and combinations thereof described in the claims.

### Solution to Problem

Representative configurations of the present disclosure for achieving the above-described objects are as follows.

According to an aspect of the present disclosure, there is provided a composition comprising a radiopaque substance whose average particle size (D50 value) is in a range of 0.1 to 1.0 micron. The radiopaque substance may be absorbed within living tissue or inflammatory lesion.

In an embodiment, the radiopaque substance may comprise one or more selected from zirconium oxide, tungsten oxide, and niobium oxide.

In an embodiment, the radiopaque substance may have an average particle size in a range of 0.1 to 0.7 microns.

In an embodiment, the radiopaque substance may have an average particle size in a range of 0.1 to 0.5 microns.

In an embodiment, absorption of the radiopaque substance may occur within one year after the composition is applied to living tissue or inflammatory lesion.

In an embodiment, absorption of the radiopaque substance may occur within six months after the composition is applied to living tissue or inflammatory lesion.

In an embodiment, absorption of the radiopaque substance may occur within four months after the composition is applied to living tissue or inflammatory lesion.

In an embodiment, absorption of the radiopaque substance may occur within one month after the composition is applied to living tissue or inflammatory lesion.

In an embodiment, the composition may be a dental composition, for example a filling material composition.

According to another aspect of the present disclosure, there is further provided a root canal filling material composition, comprising calcium hydroxide or a material that produces calcium hydroxide and the radiopaque substance.

According to yet another aspect of the present disclosure, there is further provided a permanent root canal filling material composition, comprising calcium silicate and the radiopaque substance.

According to still yet another aspect of the present disclosure, there is further provided a healing or treatment method comprising applying, to a subject, a composition comprising the radiopaque substance.

In an embodiment, the method may be a method for endodontic treatment of a tooth. In addition, the composition may be applied to a subject's root canal, such as root canal from which pulp has been removed.

According to still yet another aspect of the present disclosure, there is further provided a medical or dental use, such as for endodontic treatment, of the radiopaque substance or the composition.

In addition to those mentioned above, other additional configurations may be further included in the composition comprising the radiopaque substance according to the present disclosure without departing from the technical spirit of the present disclosure.

### Advantageous Effects of Invention

According to the present disclosure, there is provided a radiopaque substance that has improved bioabsorbability and is rapidly absorbed in living tissue such as living soft tissue or lesion site.

According to the present disclosure, there is provided a radiopaque substance with low or little biotoxicity.

According to the present disclosure, there is provided a composition comprising a radiopaque substance that is rapidly absorbed by living tissue such as living soft tissue or lesion and does not interfere with normal healing and regeneration of the living tissue.

According to the present disclosure, for cases where a temporary or permanent root canal filling material is present in living tissue, such as soft tissue or unwanted area (for example, uncontrolled inflammatory lesion) of tissue, including a case where the root canal filling material goes beyond apical foramen, there is provided a root canal filling material composition comprising a radiopaque substance that is rapidly absorbed before healing occurs and does not interfere with normal healing and regeneration of tissue.

### BRIEF DESCRIPTION OF DRAWINGS

FIG. 1 shows a radiograph of a state that is before injection of the permanent root canal filling material composition according to Comparative Example 1.

FIG. 2 shows a radiograph of a state that is immediately after injection of the permanent root canal filling material composition according to Comparative Example 1.

FIG. 3 shows a radiograph of a state that is when 8 months has elapsed after injection of the permanent root canal filling material composition according to Comparative Example 1.

FIG. 4 shows a radiograph, taken according to Experimental Example 1, of a state that is before injection of the temporary root canal filling material composition.

FIG. 5 shows a radiograph, taken according to Experimental Example 1, of a state that is immediately after injection of the temporary root canal filling material composition.

FIG. 6 shows a radiograph, taken according to Experimental Example 1, of a state that is immediately after the temporary root canal filling material composition has been replaced with the permanent root canal filling material composition, wherein the replacement is done 10 days after injection of the temporary root canal filling material composition.

FIG. 7 shows a radiograph, taken according to Experimental Example 2, of a state that is before injection of the permanent root canal filling material composition.

FIG. 8 shows a radiograph, taken according to Experimental Example 2, of a state that is immediately after injection of the permanent root canal filling material composition.

FIG. 9 shows a radiograph, taken according to Experimental Example 2, of a state that is when 1 month has elapsed after injection of the permanent root canal filling material composition.

FIG. 10 shows a radiograph, taken according to Experimental Example 2, of a state that is when 10 months has elapsed after injection of the permanent root canal filling material composition.

FIG. 11 shows powder compositions used for cytotoxicity tests. Composition 1 is a powder composition in which a calcium trisilicate (C₃S) compound and zirconium oxide having an average particle size (D50 value) of 1.5 microns are mixed in a weight ratio of 50:50; Composition 2 is a powder composition according to an embodiment of the present disclosure, that is, a powder composition in which a calcium trisilicate (C₃S) compound and zirconium oxide having an average particle size (D50 value) of 0.5 microns are mixed in a weight ratio of 50:50; and Composition 3 is a powder composition in which a calcium trisilicate (C₃S) compound and barium sulfate are mixed in a weight ratio of 80:20.

FIG. 12 shows results obtained by dispensing each of the eluates of Samples 1, 2, and 3, which have been prepared in Experimental Example 3.1, performing incubation for 24 hours, 48 hours, and 72 hours, respectively, and measuring absorbance at 590 nm to evaluate cell viability. The results obtained by performing the incubation for 24 hours, 48 hours, and 72 hours were indicated as D1, D2, and D3, respectively, and the control group was indicated as Con.

FIG. 13 shows a schematic view of a method for evaluating cell migration ability.

FIG. 14 shows results obtained by evaluating effects (wound healing rates) of the eluates of Samples 1, 2, and 3, which have been prepared in Experimental Example 3.1, on cell migration ability. The control group was indicated as Con.

### DETAILED DESCRIPTION

In the detailed description of the present disclosure to be described later, reference is made to the accompanying drawings that show, by way of illustration, specific embodiments in which the present disclosure may be practiced. These embodiments are described in sufficient detail to enable those skilled in the art to practice the present disclosure. It is to be understood that various embodiments of the present disclosure, although different from each other, are not necessarily mutually exclusive. For example, specific shapes, structures, and characteristics described herein may be implemented in such a manner that changes are made from one embodiment to another, or two or more embodiments are combined, without departing from the spirit and scope of the present disclosure. It is also to be understood that location or arrangement of individual components in each embodiment may vary without departing from the spirit and scope of the present disclosure. Therefore, the following detailed description is not intended to limit the present disclosure, and the scope of the present disclosure should be construed as encompassing the scope of the appended claims and all equivalents thereof. In the drawings, like reference numbers indicate the same or similar elements throughout several views.

Hereinafter, various preferred embodiments of the present disclosure will be described in detail with reference to the accompanying drawings in order to enable those skilled in the art to easily practice the present disclosure.

### PREFERRED EMBODIMENTS OF PRESENT DISCLOSURE

1) Radiopaque Substance

The radiopaque substance of the present disclosure has an average particle size (D50 value) adjusted so that the substance can be rapidly absorbed in living tissue such as living soft tissue or inflammatory lesion, and thus exhibits improved bioabsorbability. The average particle size D50 means a particle size (that is, particle diameter), which corresponds to 50% in volume-based cumulative particle diameter distribution, and may also be referred to as a median diameter. D50 is commonly used to indicate an average particle size of powder.

In general, the radiopaque substance may be used alone or in the form of a composition together with other components when reading is required to observe application position, healing progress, or the like in a case where application to a living body is performed. The radiopaque substance according to the present disclosure may be used for a medical or dental composition to be applied to a living body. For example, the radiopaque substance may be used for a filling material composition for endodontic treatment that is a branch of dentistry. The radiopaque substance may be included in a composition together with a dental filling material and/or a dentally acceptable excipient. In a case where a radiopaque substance is used for a filling material composition for endodontic treatment, the radiopaque substance, which unintentionally goes beyond apical foramen in the course of endodontic treatment, preferably needs to be absorbed and removed before healing occurs so as not to interfere with subsequent normal healing and regeneration of tissue. The radiopaque substance of the present disclosure may have a size suitable for being eaten by macrophages, osteoclasts, odontoclasts, and the like in a dental root region, thereby exhibiting improved bioabsorbability.

The radiopaque substance of the present disclosure may have an average particle size (D50 value) of 1.0 micron or smaller. In a case where the radiopaque substance has an average particle size of greater than 1.0 micron, the radiopaque substance may remain around a target area to be applied or in other areas (for example, apical root area or outside apical foramen), thereby interfering with formation of normal tissue (for example, hard tissue) or restoration of inflammatory lesion site, wherein, in particular, the remaining radiopaque substance may be continuously observed on radiographs, which leads to misunderstanding in subsequent diagnosis or treatment or leaves a trace of inappropriate treatment. In addition, the radiopaque substance of the present disclosure may have an average particle size (D50 value) of 0.1 microns or greater. In a case where the radiopaque substance of the present disclosure has an average particle size of smaller than 0.1 microns, a problem may occur in which the radiopaque substance enters normal cells such as osteogenic cells and periodontal ligament cells.

For this reason, the radiopaque substance may preferably have an average particle size (D50 value) in a range of 0.1 to 1.0 micron (equal to or greater than 0.1 microns to equal to or smaller than 1.0 micron), with a range of 0.1 to 0.7 microns (equal to or greater than 0.1 microns to equal to or smaller than 0.7 microns) being more preferred. Even more preferably, the average particle size may be in a range of 0.1 to 0.5 microns (equal to or greater than 0.1 microns to equal to or smaller than 0.5 microns). In addition, yet even more preferably, the radiopaque substance may have an average particle size in a range of 0.3 to 0.7 microns (equal to or greater than 0.3 microns to equal to or smaller than 0.7 microns) or 0.4 to 0.6 microns (equal to or greater than 0.4 microns to equal to or smaller than 0.6 microns).

In other embodiments, the radiopaque substance may have an average particle size of equal to or smaller than 1.0, 0.9, 0.8, 0.7, 0.6, or 0.5 microns. In addition, the radiopaque substance may have an average particle size of equal to or greater than 0.1, 0.2, 0.3, 0.4, or 0.5 microns. In addition, the radiopaque substance may have an average particle size that is any combination of numerical ranges selected from equal to or greater than 0.1, 0.2, 0.3, 0.4, or 0.5 microns and equal to or smaller than 1.0, 0.9, 0.8, 0.7, 0.6, or 0.5 microns.

The radiopaque substance is absorbed within at least one year from a time point when it has been applied or a time point when it has gone beyond apical foramen, so that the radiopaque substance does not remain in a significant amount in surrounding areas. Preferably, the radiopaque substance may be effectively absorbed within at least 6 months. More preferably, the radiopaque substance may be effectively absorbed within at least 4 months. In an embodiment, it was identified that the radiopaque substance was absorbed within at least one month based on a time point when it has been applied and did not remain in a significant amount in surrounding areas (see Experimental Example 2 and the like). Absorption timing may be adjusted by changing a particle size of the radiopaque substance within the above-described range of its average particle size.

The radiopaque substance may comprise at least one selected from zirconium oxides (for example, ZrO₂), tungsten oxides (for example, tungsten(III) oxide (W₂O₃), tungsten dioxide (WO₂), tungsten trioxide (WO₃), and calcium tungstate (CaWO₄)), and niobium oxides (for example, niobium monoxide (NbO), niobium dioxide (NbO₂), and niobium pentoxide (Nb₂O₅)). Preferably, the radiopaque substance may comprise zirconium oxide (ZrO₂).

A (average) particle size of the radiopaque substance may be adjusted by conventional methods known in the art. Specifically, the particle size may be decreased through a method of adjusting a size of heavy metal, such as wet nano grinding and air jet mill pulverization. In addition, among by-products obtained in the course of making radiopaque substances having a conventional particle size, particles having a small size, specifically, belonging the above-mentioned micron range, may be collected and provided. In addition, any method of decreasing a size of particles may be utilized without particular limitation as long as it allows the particle size to fall within a certain range.

A (average) particle size of the radiopaque substance may be measured by conventional methods known in the art. For example, the methods may include, but are limited to, a method using a laser diffraction scattering type particle size distribution measuring device and the like. Specifically, it is possible to measure a particle size or particle size distribution of a sample by dispersing the sample in a liquid phase and using laser diffraction. According to an embodiment, a particle size or particle size distribution of a sample is measured by dissolving the sample in an insoluble solvent, performing homogenization for 5 to 10 minutes by ultrasonic waves, and then using a particle size analyzer (PSA).

An amount of the radiopaque substance according to the present disclosure may be appropriately selected and used by those skilled in the art depending on properties and amounts of components to be mixed with the radiopaque substance. It may be preferred that the radiopaque substance comprises at least 20% by weight or greater based on the total amount of a composition. This is because in a case where the radiopaque substance is included in an amount of smaller than the above-mentioned weight percent, it may be difficult to properly observe the radiopaque substance on radiographs after procedure. It may be more preferred that the radiopaque substance comprises 20 to 75%, 20 to 65%, 30 to 65%, 30 to 75%, or 30 to 55% by weight based on the total amount of a composition.

Specifically, preference for an amount of the radiopaque substance may differ between temporary root canal filling materials and permanent root canal filling materials. For a temporary root canal filling material composition, a role of calcium hydroxide is important, and thus it may be preferred to ensure that calcium hydroxide comprises 30% by weight or greater based on the total amount of a composition. An amount of the radiopaque substance may be selected in a range of 30 to 65% by weight based on a total root canal filling material composition. A radiopaque substance having a relatively low molecular weight may need to be included in a higher amount than a radiopaque substance having a high molecular weight, to achieve similar radiopacity thereto. An exact amount of a radiopaque substance may be determined by proper flowability of a temporary root canal filling material composition. Typical preferred flowability for a temporary root canal filling material composition is in a range of 10 mm to 25 mm. On the other hand, it may be preferred that a permanent root canal filling material is observed more clearly on radiographs. For example, it is preferred to have radiopacity of 3 mm or higher based on an aluminum wedge. To this end, in a case where a radiopaque substance such as zirconium oxide is included alone, it may comprise 30% or greater based on the total amount of a composition.

2) Temporary Root Canal Filling Material Composition

The radiopaque substance of the present disclosure may be included in a temporary root canal filling material composition. A temporary root canal filling material composition, which is also called temporary intracanal medicament, can be applied to root canal for a predetermined period of time so that invasion or propagation of bacteria is prevented, and bacterial endotoxins are neutralized to suppress formation of apical lesions.

The temporary root canal filling material composition according to an embodiment may comprise, together with the radiopaque substance of the present disclosure, calcium hydroxide or a material (for example, calcium oxide) that produces calcium hydroxide through a hydration reaction at a level usable in a human body to control inflammation in or around root canal. Such ingredients may be provided in powder form. As calcium hydroxide or calcium oxide has a smaller particle size, a better reaction with water (for example, water in a human body) occurs. However, in a case where the particle size of calcium hydroxide or calcium oxide is decreased to equal to or smaller than several tens of nanometers, a temporary root canal filler composition may have increased viscosity, which may be disadvantageous when the composition needs to be removed at a later time. Calcium hydroxide plays an important role in a temporary root canal filling material composition, and thus it may be preferred that calcium hydroxide comprises 30% or higher based on the total weight of the composition.

As a material that produces calcium hydroxide when mixed with water, portland cement or pozzolan cement, which contains a calcium silicate compound as a main component, may be mentioned. The present inventor(s) hold several patents (or patent applications) by themselves or through companies, to which the inventor(s) belongs, regarding various inventions by which such cements have been developed for dental use. For this purpose, reference may be made to Korean Patent Application Nos. 10-2008-0038387, 10-2012-0028458, 10-2013-0112165, 10-2014-0032686 and 10-2014-0122694, each of the disclosures of which is to be considered as incorporated herein in its entirety. Therefore, a component necessary for the temporary root canal filling material composition according to an embodiment of the present disclosure, that is, a material that produces calcium hydroxide through a hydration reaction may be prepared by those skilled in the art using techniques disclosed in the above-described patent documents or slightly applying such techniques.

The portland cement reacts with water to produce hydrous calcium silicate and calcium hydroxide so that calcium hydroxide tends to occupy a significant portion of the surface after curing, which is not preferable. Therefore, it may be more preferred that the portland cement comprises a pozzolanic material capable of converting calcium hydroxide, which is present on the surface after curing, into hydrous calcium silicate that is neutral and stable in a human body.

For ease of application, the temporary root canal filling material composition may further comprise a component that exists as a liquid at room temperature. Such a liquid may be selected from polyhydric alcohols such as propylene glycol (PG) and polyethylene glycol (PEG), which have higher viscosity than water, or liquids having viscosity similar to water, such as dimethyl sulfoxide (DMSO), N-methyl-2-pyrrolidone (NMP), and diethylene glycol monoethyl ether (DEGEE); however, any liquid may be used as long as it can be used in a human body, can be mixed with a strongly basic substance, can be easily mixed with water, and can faciliate penetration of the composition.

In an embodiment, the liquid may comprise one or more selected from the group consisting of PG, PEG, DMSO, NMP, and DEGEE. In addition, in order for a combination, which comprises at least one selected from water, DMSO, NMP, and DEGEE, to be rapidly absorbed in a human body so as not to interfere with healing of normal tissue, it may be preferred that the combination accounts for 70% or higher of the total weight of the solution. As an example of the temporary root canal filling material composition, reference may be made to Korean Patent No. 10-2233620, which is held by the present inventor(s) or companies, to which the inventor(s) belongs, and the disclosure of which is to be considered as incorporated herein in its entirety. Therefore, the temporary root canal filling material composition according to an embodiment of the present disclosure may be prepared by those skilled in the art using a technique disclosed in the patent or slightly applying such a technique.

In an embodiment, the temporary root canal filling material composition comprises, as powder components, calcium hydroxide or a material that produces calcium hydroxide and the radiopaque substance according to the present disclosure, comprises, as a liquid component, at least one of DMSO, NMP, and DEGEE, and comprises a thickener, wherein the thickener may be included in an amount of higher than 0% to equal to or smaller than 10% based on a weight of the calcium hydroxide, and the at least one of DMSO, NMP, and DEGEE may account for 70% or higher of a weight of the liquid component.

A thickener may be further included in the temporary root canal filling material composition since proper flowability is required for the composition to be applied to every nook and cranny of root canal. In particular, it may be preferred to add a thickener in a case of using a liquid having relatively low viscosity, such as DMSO, NMP, DEGEE, or water. It may be preferred that such a thickener needs to be easily dissolved in water and does not affect ionization of calcium hydroxide. Specifically, the thickener may comprise at least one of a cellulose derivative such as methyl cellulose, hydroxy ethyl cellulose, hydroxypropyl methyl cellulose (HPMC), and carboxy methyl cellulose, polyvinyl alcohol (PVA), and polyvinylpyrrolidone (PVP). It may be preferred that for proper viscosity of the composition, the thickener is included in an amount of 10% or lower based on a weight of calcium hydroxide. In addition, for the above-mentioned thickeners, rather than using only one thickener, it may be more effective to use an appropriate combination thereof depending on selection of those skilled in the art.

In addition, at least one of polyols such as xylitol, erythritol, and sorbitol may be included as the thickener. At least one of bentonite, hectorite, and expandable clay may be included as the thickener. At least one of water-soluble chitin and a chitosan derivative may be included as the thickener.

3) Permanent Root Canal Filling Material Composition

The radiopaque substance of the present disclosure may be included in a permanent root canal filling material composition. The permanent root canal filling material composition is used, after removal of infectious agents in root canal, to seal an inside of the root canal so that secondary infection is suppressed and healing is promoted. The permanent root canal filling material composition may comprise, together with the radiopaque substance according to the present disclosure, a bioceramic root canal filling material containing a compound (for example, a hydraulic compound such as calcium silicate or calcium aluminate) that reacts with moisture present in or around root canal and hardens therein to seal an inside of the root canal. As the compound, calcium silicate having low toxicity and high bioactivity in vivo is preferably used, but the compound is not limited thereto.

Calcium hydroxide, which is produced by hydration of the above-described calcium silicate component, is initially effective and safe in root canal. However, calcium hydroxide may react with collagen, which forms tooth structure, and weaken the tooth structure in a case of being present in the root canal for a too long time. Thus, it may be preferred to further comprise a pozzolanic material that consumes calcium hydroxide. As the pozzolanic material, amorphous silica such as fumed silica, precipitated silica, and colloidal silica may be selected; and in addition, metakaolin, diatomaceous earth, and swelling clay (for example, swelling phyllosilicate such as bentonite, hectorite, and synthetic swelling clay) may be appropriately used.

The permanent root canal filling material composition may further comprise a liquid component so that the composition is provided in a pre-kneaded state. Examples of the liquid component include, but are limited to, dimethyl sulfoxide (DMSO), N-methyl-2-pyrrolidone (NMP), diethylene glycol monoethyl ether (DEGEE), polyethylene glycol (PEG), polysorbate, and trimethylene glycol.

The permanent root canal filling material composition may further comprise a thickener to adjust viscosity of the liquid component. For the thickener, reference is made to the description thereof for the temporary root canal filling material composition.

In an embodiment, there is provided a single-paste type hydraulic root canal filling material composition, comprising a calcium silicate component, a liquid component, and the radiopaque substance of the present disclosure. The single paste-type hydraulic root canal filling material composition may comprise DMSO as a liquid component for rapid absorption in a human body. DMSO may account for 70% or higher of a weight of the liquid component of the composition. In this case, the liquid component may further comprise at least one selected from the group consisting of water, ethanol, PEG, and DEGEE in an amount of 30% or lower of a weight of the liquid component.

4) Application Method or Use of Composition According to Embodiment of Present Disclosure

There is provided a method comprising applying, to a subject in need of dental treatment, a composition, temporary root canal filling material composition, or permanent root canal filling material composition, which comprises the above-described radiopaque substance, or a dental use of such a composition. In an embodiment, the composition may be applied to root canal of a tooth. In another embodiment, the composition may be applied to root canal from which pulp tissue has been removed. In an embodiment, the method or use may relate to dental treatment commonly used in dentistry, such as endodontic treatment, or uses therefor. In addition, the method or use may comprise other steps or applications commonly used in dental treatment.

### MODE FOR CARRYING OUT INVENTION

Hereinafter, the present disclosure will be described in more detail by way of the following examples. However, the following examples are only for illustrating the present disclosure, and the scope of the present disclosure is not limited only to these examples.

In the following Comparative Examples and Examples, measurement of an average particle size (D50 value) was performed by dissolving a sample in an insoluble solvent, subjecting the sample to ultrasonic homogenization in a sonicator for 5 to 10 minutes, and then using a particle size analyzer.

### Comparative Example 1. Permanent Root Canal Filling Material Composition

A permanent root canal filling material composition was prepared by mixing calcium trisilicate and zirconium oxide having an average particle size (D50 value) of 1.5 microns in a weight ratio of 50:50.

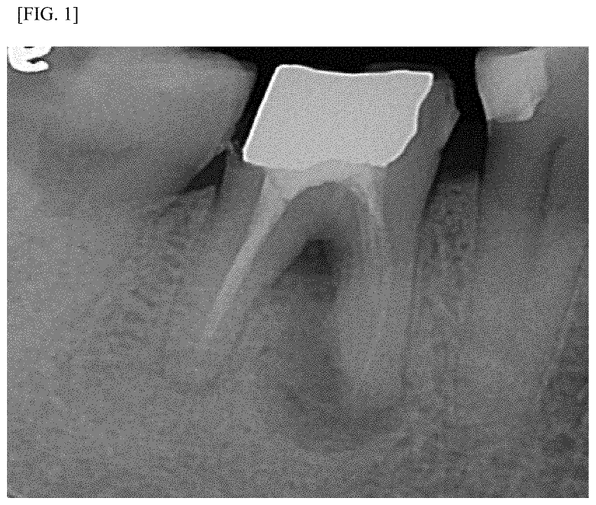

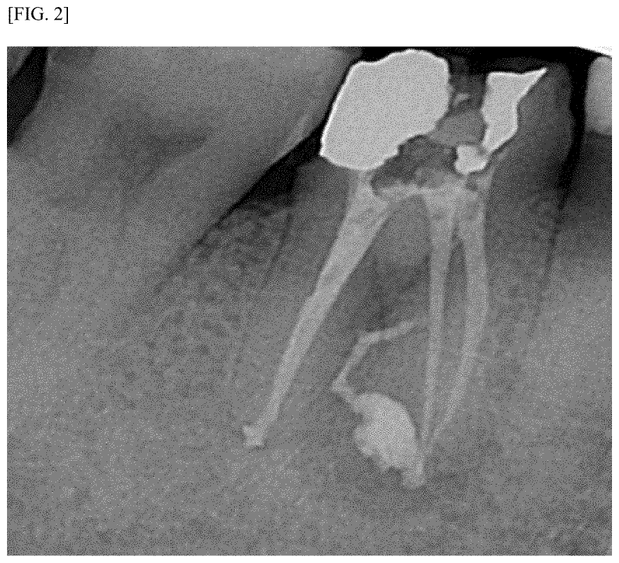

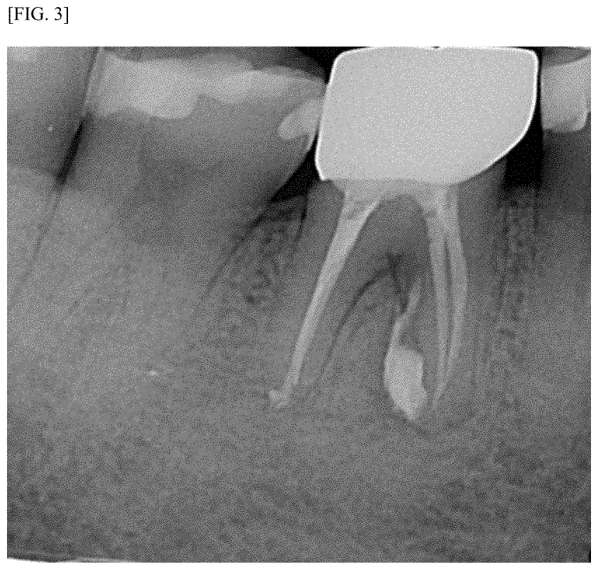

For a case where the permanent root canal filling material composition was applied to root canal and excessively injected beyond apical foramen, radiographs were taken before the injection, immediately after the injection, and when 8 months had elapsed after the injection. Referring to FIGS. 1 to 3, it is possible to identify that the filling material, which had gone beyond apical foramen, remained even after inflammation-damaged alveolar bone was completely regenerated.

### Example 1. Temporary Root Canal Filling Material Composition (PEG400+PEG200)

As a powder component, calcium hydroxide and zirconium oxide having an average particle size (D50 value) of 0.5 microns were mixed in a weight ratio of 50:50; and as a liquid component, PEG400 and PEG200 were mixed in a 1:1 ratio. Then, the powder component and the liquid component were kneaded together to have flowability of 15 mm, thereby preparing a temporary root canal filling material composition.

### Example 2. Temporary Root Canal Filling Material Composition (DMSO+HPMC)

As a powder component, calcium hydroxide and zirconium oxide having an average particle size (D50 value) of 0.5 microns were mixed in a weight ratio of 50:50; and as a liquid component, HPMC was dissolved in DMSO at 2%. Then, the powder component and the liquid component were kneaded together to have flowability of 15 mm, thereby preparing a temporary root canal filling material composition.

### Example 3. Permanent Root Canal Filling Material Composition

A permanent root canal filling material composition was prepared by mixing calcium trisilicate and zirconium oxide having an average particle size (D50 value) of 0.5 microns in a weight ratio of 50:50.

### Experimental Example 1. Absorption of Radiopaque Substance in Case where Temporary Root Canal Filling Material Composition is Excessively Injected

The temporary root canal filling material composition according to Example 2 was excessively injected beyond apical foramen during endodontic treatment. 10 days after the injection, the temporary root canal filling material composition was removed and replaced with a permanent root canal filling material composition.

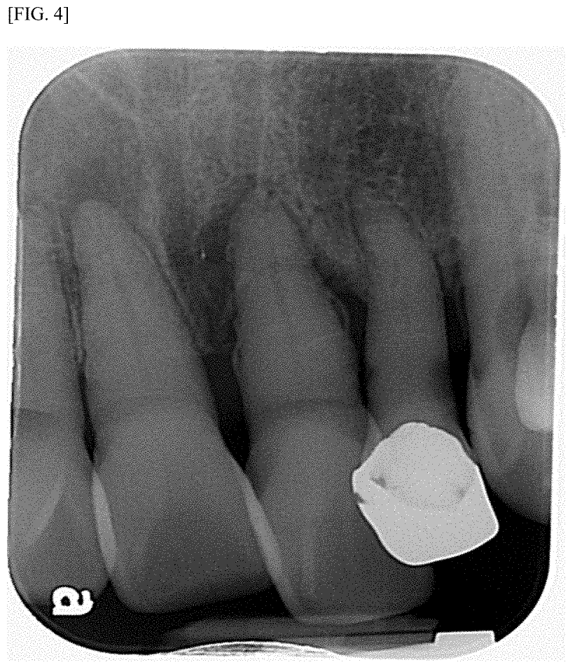

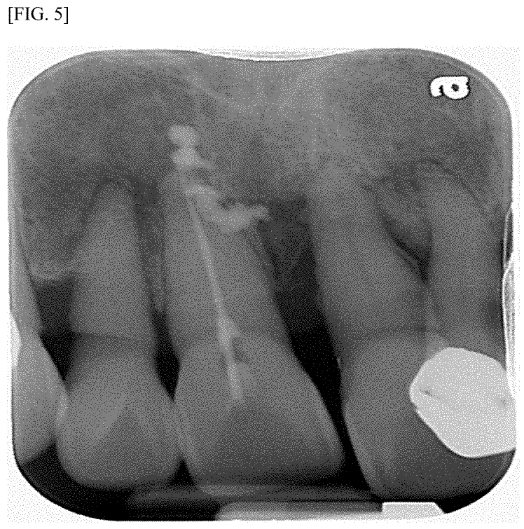

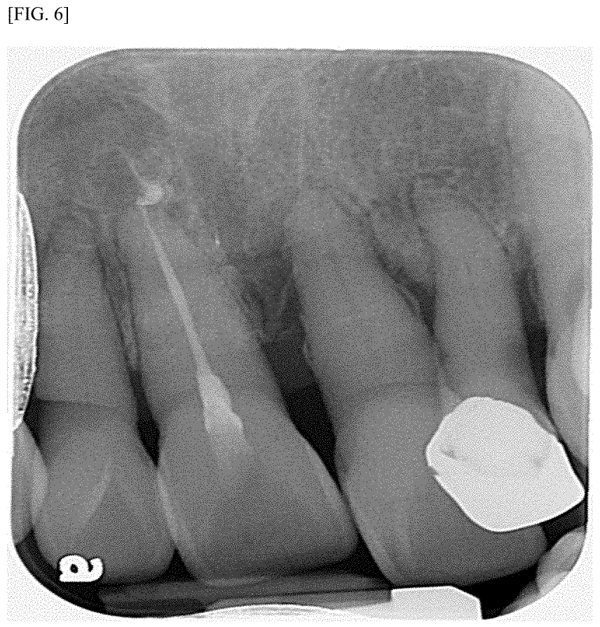

For comparison, radiographs were taken for a state that was before the injection, a state that was immediately after the injection, and a state that was immediately after the replacement with the permanent root canal filling material composition which was performed 10 days after the injection. Referring to FIGS. 4 to 6, it is possible to identify that the radiopaque substance, which had gone beyond apical foramen and entered surrounding tissue, was well absorbed.

### Experimental Example 2. Absorption of Radiopaque Substance in Case where Permanent Root Canal Filling Material Composition is Excessively Injected

The permanent root canal filling material composition according to Example 3 was kneaded with water and excessively injected beyond apical foramen during endodontic treatment. Here, a kneading ratio of the powder and the water was 100:40.

For comparison, radiographs were taken for a state that was before the injection, a state that was immediately after the injection, a state that was when 1 month had elapsed after the injection, and a state that was when 10 months had elapsed after the injection.

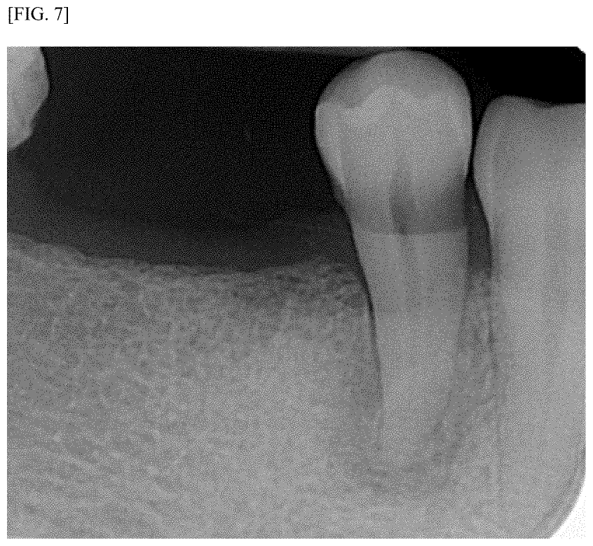

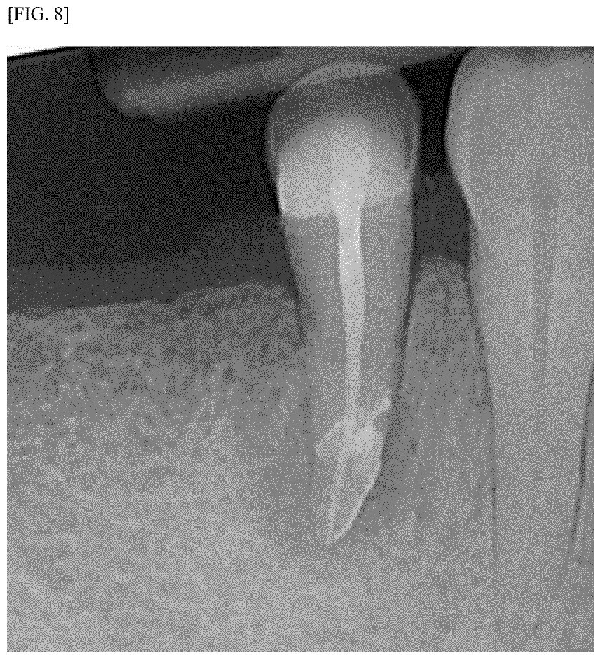

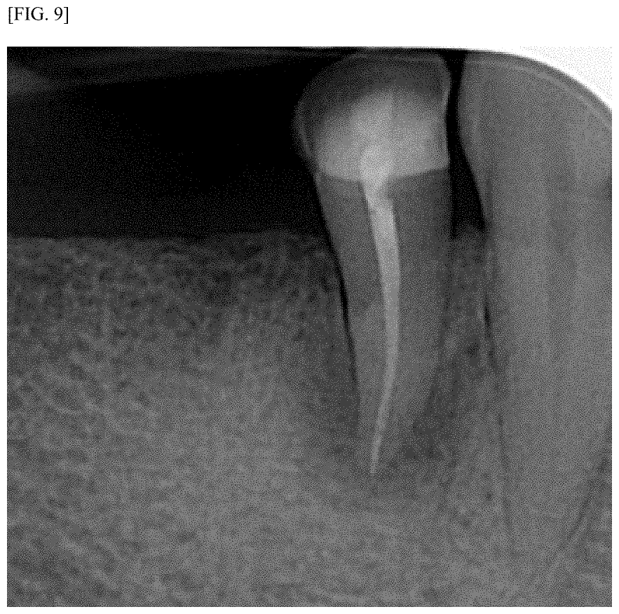

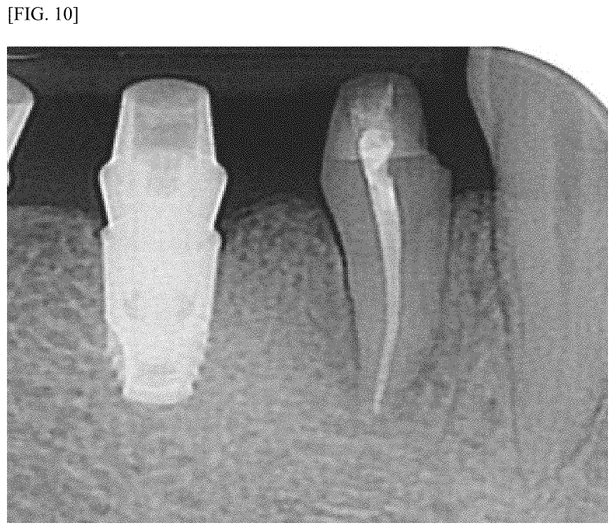

Referring to FIGS. 7 to 9, it is possible to observe that the radiopaque substance, which had passed to a lesion site, was completely absorbed when 1 month had elapsed after the injection. In addition, referring to FIG. 10, it is possible to observe that hard tissue inflammation healed well with formation of new hard tissue when 10 months had elapsed after the injection.

### Experimental Example 3. Evaluation of Cytotoxicity of Root Canal Filling Material Composition Comprising Radiopaque Substance

### Experimental Example 3.1. Cell Viability Evaluation (MTT Assay)

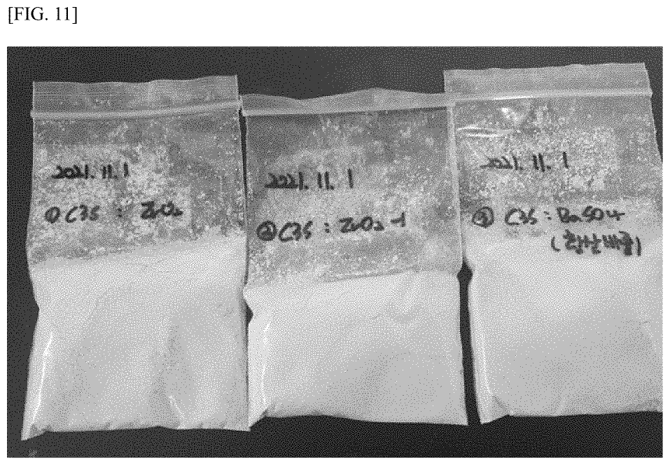

Three types of powder were prepared to evaluate biocompatibility of the radiopaque substance according to the present disclosure. Sample 1 is a powder composition in which a calcium trisilicate (C₃S) compound and zirconium oxide having an average particle size (D50 value) of 1.5 microns are mixed in a weight ratio of 50:50; Sample 2 is a powder composition in which a calcium trisilicate (C₃S) compound and zirconium oxide having an average particle size (D50 value) of 0.5 microns are mixed in a weight ratio of 50:50; and Sample 3 is a powder composition in which a calcium trisilicate (C₃S) compound and barium sulfate are mixed in a weight ratio of 80:20 (see FIG. 11). Barium sulfate was adjusted to 20% so that it has the same level of radiopacity as zirconium oxide, and then added.

Each of the powder samples prepared above was put into a mold with a thickness of 1 mm and a diameter of 5 mm, and cured in an incubator at 37° C. for 3 days to prepare a specimen. The specimen was taken out of the mold and sterilized with UV light on the front and back sides for 1 day, respectively. Then, the specimen was incubated at 37° C. for 3 days in MEM-α medium for cell culture (minimal essential medium, HyClone Laboratories, Logan, UT, USA) at a ratio of medium per surface area of 0.5 ml/cm², to prepare a sample eluate (material extract).

The sample eluate was used to perform cell viability evaluation (MTT assay). First, L929 cells (Korean Cell Line Bank) were seeded at 7×10³ cells per well in a 96-well plate, and cultured for 24 hours. Then, the medium was removed, and each sample eluate from the three types of sample as prepared above was dispensed therein at 100 ul. For each experiment group, 3 wells were prepared for experiments. After dispensing each sample eluate, culture was performed for 24 hours, 48 hours, and 72 hours, respectively. For each control group, the same amount of medium was dispensed without addition of the sample eluate. After the culture, the eluate was discarded, and treatment with 100 ul of 0.05% MTT solution was performed. Then, each well was wrapped in foil, and reaction was allowed to proceed in an incubator at 37° C. for 2 hours. After the reaction, 100 ul of dimethyl sulfoxide (DMSO) was added, and then 100 ul of the reacted solution was transferred to a new 96-well plate. Absorbance was measured at 590 nm using a spectrometer.

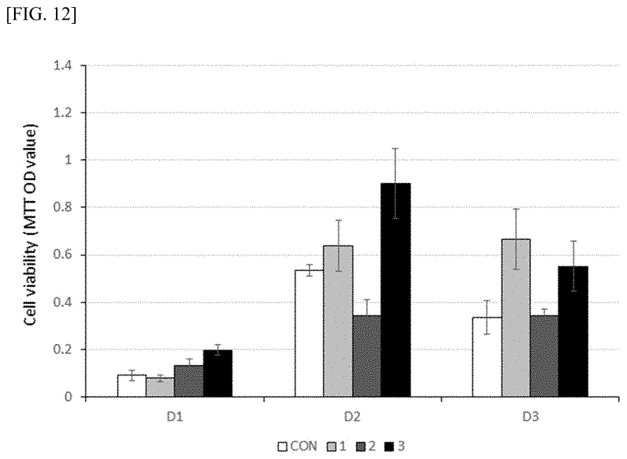

As a result of the measurement, referring to FIG. 12, the highest viability was observed in the 96-well plate cultured for 48 hours (D2), and the cell viability was generally good for the culture plate to which the sample eluate of Sample 3 was applied. However, the cell viability decreased over time in a case of the culture plate to which the sample eluate of Sample 3 was applied, whereas the cell viability was maintained or increased over time in a case of the culture plate to which the sample eluate of Sample 1 or 2 was applied. As such, it was identified that zirconium oxide, in particular, zirconium oxide (Sample 2) whose size has been adjusted according to the present disclosure shows biocompatibility comparable to Sample 3 using barium sulfate, which has been widely used in the art and has secured some degree of biocompatibility.

### Experimental Example 3.2. Evaluation of Cell Migration Ability

Evaluation of cell migration ability (scratch assay) was performed to identify how effectively cells heal pre-formed scratches in each of the sample eluates prepared in Experimental Example 3.1.

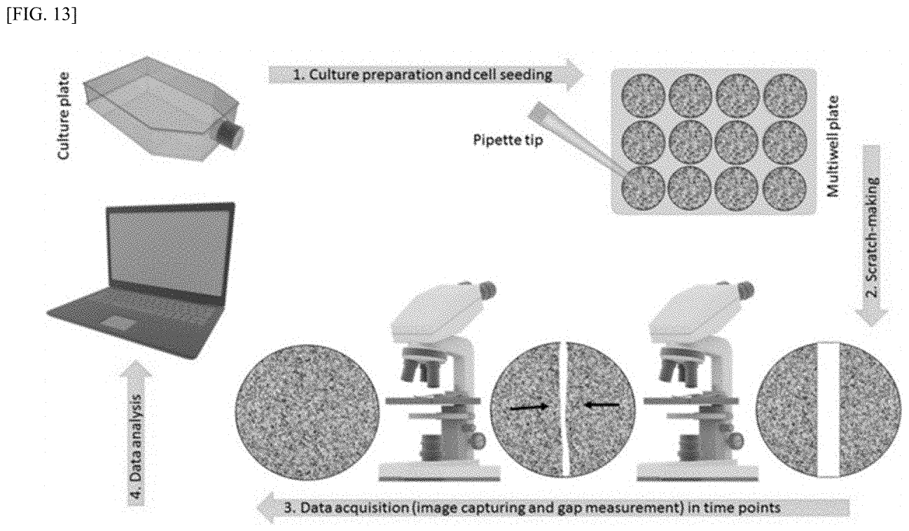

L929 cells (Korean Cell Line Bank) were seeded in a 24-well plate, and cultured in an incubator at 37° C. until a confluent monolayer was formed. When the cells filled the plate (which means 100% confluency), a sterile plastic micropipette tip or razor blade was used to make a straight line (scratch) across the cell monolayer in each well. Here, the scratch making in all wells was done by aligning the pipette's angle and applying a constant pressure to create a constant interval. After the scratch making, the cell monolayer was washed with basal medium (MEM-α, HyClone Laboratories) to remove cell debris, and the eluate of each of Samples 1, 2, and 3 prepared in Experimental Example 3.1 were added thereto. For the control group, basal medium was added thereto at an equal amount. Next, the plate was cultured in an incubator under a condition of 37° C. and 5% CO₂. While performing the culture, changes in scratch were measured by taking pictures with an optical microscope at 6 and 12 hours. Prior to that, a picture was obtained with an optical microscope at 0 hour after the scratch making. The scratched area was quantitatively calculated using the open source software ImageJ. FIG. 13 shows a schematic diagram of the method for evaluating cell migration ability. By checking whether the scratch artificially formed on the cell monolayer can be rapidly restored by surrounding cells, it is possible to determine whether the eluate affects growth of the cells (that is, whether the eluate interferes with growth of the cells).

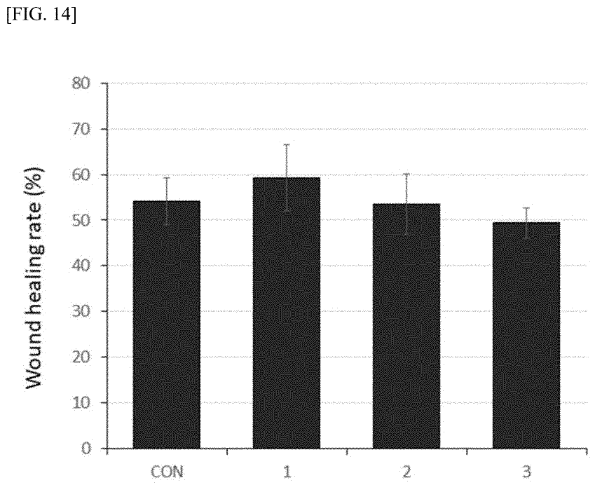

As a result of the experiment, referring to FIG. 14, there was no significant difference in cell migration ability between all experimental groups. Therefore, it is suggested that the nano zirconium oxide of Sample 2 does not interfere with healing of wounds or lesions in a case of being used as a radiopaque substance for a root canal filling material.

Statistical Analysis

Statistical analysis was performed using the Kruskal-Wallis method, which is a non-parametric method, with a significance level of 0.05 (P<0.05).

1. A composition comprising a radiopaque substance,
wherein the radiopaque substance has an average particle size (D50 value) in a range of 0.1 to 1.0 micron and can be absorbed within living tissue or inflammatory lesion.

2. The composition of claim 1,
wherein the radiopaque substance comprises at least one selected from zirconium oxide, tungsten oxide, and niobium oxide.

3. The composition of claim 1,
wherein the radiopaque substance comprises zirconium oxide.

4. The composition of claim 1,
wherein the average particle size is in a range of 0.1 to 0.7 microns.

5. The composition of claim 1,
wherein the average particle size is in a range of 0.1 to 0.5 microns.

6. The composition of claim 1,
wherein the absorption occurs within one year after the composition has been applied to the living tissue or the inflammatory lesion.

7. The composition of claim 1,
wherein the absorption occurs within 6 months after the composition has been applied to the living tissue or the inflammatory lesion.

8. The composition of claim 1,
wherein the absorption occurs within 4 months after the composition has been applied to the living tissue or the inflammatory lesion.

9. The composition of claim 1,
wherein the radiopaque substance comprises 30 to 65% by weight based on a total amount of the composition.

10. A root canal filling material composition, comprising:
calcium hydroxide or a material that produces calcium hydroxide; and
the composition comprising a radiopaque material of claim 1.

11. The root canal filling material composition of claim 10,
wherein the composition comprises the calcium hydroxide or the material that produces calcium hydroxide as a powder component, and
at least one of water, dimethyl sulfoxide (DMSO), N-methyl-2-pyrrolidone (NMP), and diethylene glycol monoethyl ether (DEGEE) as a liquid component.

12. A permanent root canal filling material composition, comprising:
calcium silicate; and
the composition comprising a radiopaque substance of claim 1.

13. A method comprising applying the composition of claim 1 to a subject in need of dental treatment.

14. The method of claim 13,
wherein the composition is applied to root canal of a tooth.

15. A dental use of the composition of claim 1.

16. The dental use of claim 15,
wherein the composition is used for endodontic treatment of a tooth.
